(19) United States
(12) Patent Application Publication
(10) Pub. No.: US 2011/0144404 A1
(43) Pub. Date: Jun. 16, 2011

(54) MTO Feed Purification

(75) Inventor: Joseph A. Montalbano, Bartlett, IL (US)

(73) Assignee: UOP LLC, Des Plaines, IL (US)

(21) Appl. No.: 12/637,243

(22) Filed: Dec. 14, 2009

(51) Int. Cl. C07C 1/24

(52) U.S. Cl. 585/639; 585/638

(57) ABSTRACT

A process is presented for improving the quality of an oxygenate feedstream to an oxygenate to olefins conversion reactor. The process includes passing the feedstream to a fractionation column having a sufficient number of trays to remove the sodium in the form of sodium hydroxide. The fractionation column is also sized to have a sufficient number of trays to minimize the amount of oxygenates being passed out with the removed sodium hydroxide.

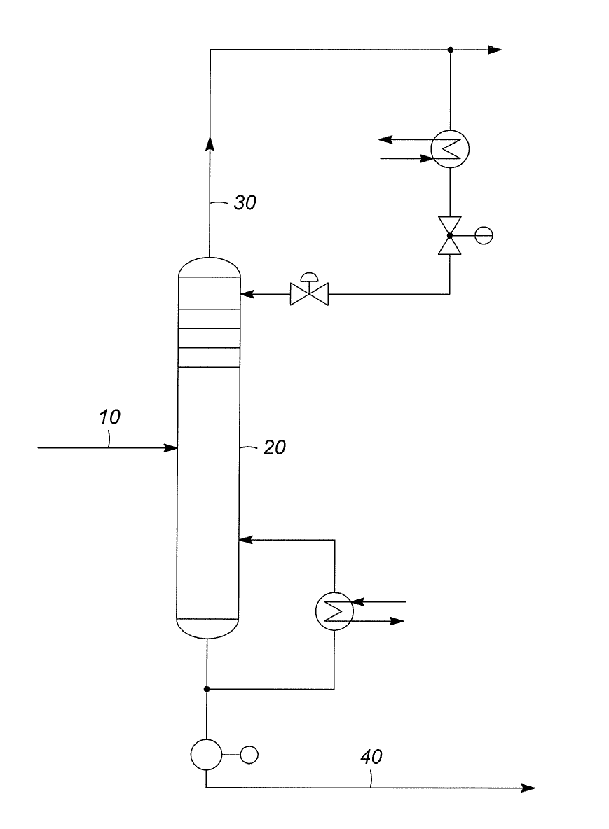

### FIELD OF THE INVENTION

The field of the invention is the process for conversion of methanol to olefins. In particular, the methanol to olefins process uses a catalyst that is susceptible to deactivation due to impurities in the feed.

### BACKGROUND OF THE INVENTION

The traditional method of olefin production is the cracking of petroleum feedstocks to olefins. The cracking of petroleum feedstocks is done through catalytic cracking, steam cracking, or some combination of the two processes. The olefins produced are generally light olefins, such as ethylene and propylene. There is a large market for the light olefin products of ethylene and propylene. As petroleum feedstocks from crude oil face increasing prices it is advantageous to provide for other sources of ethylene and propylene. It is also known that olefins can be produced from oxygenates. The most common conversion of oxygenates to olefins is the production of light olefins from methanol, wherein methanol can be produced from other sources, including biomass, and natural gas.

The process of converting oxygenates to olefins is an important process for utilizing oxygenates, such as methanol, and converting them to higher value products such as monomers for plastics, such as ethylene and propylene. The process of converting oxygenates to olefins is a catalytic process, and the catalyst is usually a molecular sieve catalyst. Among the molecular sieves that are useful for the catalytic process are ZSM-type molecular sieves, but more particularly, it has been found that silico-aluminophosphate (SAPO) molecular sieves work well in the process.

SAPOs are synthesized by forming a mixture containing sources of silicon, aluminum, and phosphorus mixed with an organic template, and then crystallizing the molecular sieve at reaction conditions. Many factors affect the form the molecular sieve takes, including the relative amounts of the different components, the order of mixing, the reaction conditions, e.g. temperature and pressure and the choice of organic template.

However, MTO catalysts are expensive and susceptible to deactivation. Protection of MTO catalysts during the MTO process is important to enable a viable commercial process. A particularly potent poison for MTO catalysts is NaOH.

### SUMMARY OF THE INVENTION

In the methanol to olefins process, an oxygenate feedstream is produced that contains sodium hydroxide (NaOH). The feedstream is passed through a flash drum to remove a portion of the water from the feedstream. The process allows too much NaOH to pass to an olefins conversion reactor, and can deactivate the catalyst in the olefins conversion reactor. The present invention includes passing the oxygenate to a feed fractionation column, thereby creating an overhead oxygenate feedstream to the olefins conversion reactor. The oxygenate feedstream has a reduced NaOH content, thereby allowing for longer life of the olefins conversion catalyst. The feed fractionation column has at least three trays in the rectifying section to remove the NaOH. In one embodiment, the feed fractionation column includes at least six trays in the stripping section to reduce the methanol in the bottoms stream.

Other objects, advantages and applications of the present invention will become apparent to those skilled in the art from the following detailed description and drawing.

### BRIEF DESCRIPTION OF THE DRAWING

FIGURE is a diagram of the invention.

### DETAILED DESCRIPTION OF THE INVENTION

The methanol to olefins process (MTO) is an important process, and uses an expensive catalyst for converting oxygenates to more valuable olefins. The catalyst is susceptible to deactivation by contaminants such as metals that are in the feed. Metals found in the feed include sodium in the form of sodium hydroxide (NaOH). Removal of the contaminants without sacrificing the loss of the methanol is important. Prior methods for the removal of contaminants used a feed vaporization drum. The feed is vaporized leaving the metals behind, however, the system still allows for NaOH to enter the reactor and deactivate the catalyst.

The current MTO method for passing a feedstream to the MTO reactor is to pass the feed stream to a feed vaporization drum, and can include an oxygenate stripper. The overhead stream is then passed to the MTO reactor. This process allows sodium hydroxide (NaOH) to pass to the MTO reactor. The sodium deactivates the MTO catalyst, causing shorter cycle times and requiring more frequent regeneration, and replacement, of the MTO catalyst.

The present invention provides a new process for removing catalyst contaminants from the feed to an oxygenate conversion reactor. The main component of the oxygenate feedstream is methanol, and the oxygenate conversion reactor is usually referred to as an MTO reactor. The process, as shown in the Figure, involves passing an oxygenate feedstream 10 to a feed fractionation column 20, thereby creating an overhead oxygenate reactor feedstream 30 and a feed fractionation bottoms stream 40. The oxygenate reactor feedstream 30 is passed to the MTO reactor for converting oxygenates to olefins. The bottoms stream 40 is then passed to a quench tower comprising the any alkali contaminants, and in particular the sodium hydroxide. The caustic can then be used to neutralize acetic acid from the reactor.

In one embodiment, the process further comprises passing the feedstream to an oxygenate stripper, thereby creating an intermediate oxygenate feedstream. The oxygenate stripper is to reduce the water content of the oxygenate feedstream, thereby reducing the energy needs of downstream units. The intermediate oxygenate feedstream is then passed to the feed fractionation column 20.

The process can further comprise passing the feed fractionation bottoms stream to a side stripping column. The side stripping column generates a side stripping column overhead and a side stripping column bottoms stream. The process can further include passing the side stripping column overhead back to the feed fractionation column as a vapor feedstream at a feed location below the oxygenate feedstream inlet to the feed fractionation column. The side stripping column bottoms stream is passed to the quench tower. The side stripping column bottoms stream can be used to neutralize the acetic acid generated by the oxygenate conversion reactor.

The feed fractionation column and side stripper have 3 purposes. The first is to reject sodium ions, in particular in the form of sodium hydroxide, the second objective is to limit the oxygenate in the bottoms streams, and the third objective is to minimize energy use.

The primary oxygenate is methanol, and the separation in the fractionation columns is complicated by the azeotrope formed with the water, methanol and dimethyl-ether (DME) feedstream.

The feed fractionation column is the primary column for preparing the feed to the MTO reactor. The column is designed to minimize the number of trays to keep the column height down, with a reflux flow designed to achieve approximately 1.5 liter/min per cm (1 GPM/inch) of weir length. There need to be 3 or more trays above the oxygenate feed entry to remove the NaOH, and there need to be at least 3 to 6 trays below the feed entry to reduce the methanol concentration in the feed fractionation bottoms stream. The feed fractionation bottoms stream can be passed to the side stripper for further recovery of methanol from the feed fractionation bottoms stream. The side stripper is smaller than the feed fractionation column and can be operated at a higher reflux to feed ratio and at a higher reboiler duty to feed rate, since the feed to the side stripper is much smaller than the feed to the feed fractionation column. Thus, both capital and operating costs are lowered with a side stripper.

The process of the present invention is to remove the sodium contaminants from the MTO feedstream while minimizing energy use. The process comprises passing a methanol stream to a feed vaporization column to remove water, creating a methanol feedstream and a waste water stream. The waste water stream can be passed to a quench tower. The methanol feedstream 10 is passed to a feed fractionation column 20, wherein the rectifying section of the fractionation column 20 includes at least three trays. The feed fractionation column 20 generates an overhead methanol reactor feed stream 30, and a feed fractionation bottoms stream 40. A portion of the feed fractionation bottoms stream 40 is passed to a side stripping column, with the remainder of the bottoms stream being passed to the quench tower. The side stripping column can be operated at a higher reflux to feed rate and a higher reboiler duty to feed rate due to the smaller flow and smaller column. The side stripping column further recovers methanol from the fractionation column bottoms stream. The side stripping column creates a side stripping overhead stream and a side stripping bottoms stream. The side stripping overhead stream is passed back to the feed fractionation column 20 at an inlet point below the stripping section of the fractionation column 20. The side stripping bottoms stream is passed to the quench tower. The bottoms streams containing NaOH can be used to neutralize the acetic acid waste generated by the MTO reactor.

While the invention has been described with what are presently considered the preferred embodiments, it is to be understood that the invention is not limited to the disclosed embodiments, but it is intended to cover various modifications and equivalent arrangements included within the scope of the appended claims.

1. A process for purifying an oxygenate to olefins feed comprising:
passing an oxygenate feed stream to a feed fractionation column, thereby creating an overhead oxygenate reactor feed stream and a feed fractionation bottoms stream comprising sodium hydroxide; and
passing the oxygenate reactor feed stream to an oxygenate to olefins conversion reactor.

2. The process of claim 1 wherein the oxygenate is methanol.

3. The process of claim 1 further comprising passing the feed stream to an oxygenate stripper, prior to passing the oxygenate feed stream to the feed fractionation column, thereby creating an oxygenate feed stream with reduced water content.

4. The process of claim 1 further comprising passing the feed fractionation bottoms stream to a quench tower.

5. The process of claim 1 further comprising passing the feed fractionation bottoms stream to a side stripping column, thereby creating a side stripping column overhead and a side stripping column bottoms stream.

6. The process of claim 4 wherein the side stripping column bottoms stream is used to neutralize acetic acid generated by the oxygenate reactor.

7. The process of claim 6 further comprising passing the side stripping column overhead to the feed fractionation column.

8. The process of claim 6 further comprising passing the side stripping column bottoms stream to a quench tower.

9. The process of claim 6 wherein the side stripping column bottoms stream is used to neutralize acetic acid generated by the oxygenate conversion reactor.

10. A process for purifying a methanol to olefins feed comprising:
passing a methanol stream to feed vaporization drum, thereby creating the methanol feed stream;
passing the methanol feed stream to a feed fractionation column, thereby creating an overhead methanol reactor feed stream and a feed fractionation bottoms stream comprising sodium hydroxide; and
passing the methanol reactor feed stream to a methanol to olefins conversion reactor.

11. The process of claim 10 further comprising passing the feed fractionation bottoms stream to a quench tower.

12. The process of claim 11 wherein the feed fractionation bottoms stream is used to neutralize acetic acid generated by the oxygenate conversion reactor.

13. The process of claim 10 further comprising passing the feed fractionation bottoms stream to a side stripping column, thereby creating a side stripping column overhead and a side stripping column bottoms stream.

14. The process of claim 13 further comprising passing the side stripping column overhead to the feed fractionation column.

15. The process of claim 13 further comprising passing the side stripping column bottoms stream to a quench tower.

16. The process of claim 13 wherein the side stripping column bottoms stream is used to neutralize acetic acid generated by the oxygenate reactor.

17. The process of claim 10 further comprising
passing the methanol feed stream to an oxygenate stripper prior to passing the methanol feed stream to the feed fractionation column, thereby creating a methanol feed stream having a reduced water and metals content.
